(19) United States
(12) Patent Application Publication
(10) Pub. No.: US 2017/0348176 A1
(43) Pub. Date: Dec. 7, 2017

(54) High Performance Free Rolling Cable Transmission

(71) Applicant: Massachusetts Institute of Technology, Cambridge, MA (US)

(72) Inventors: Hugh M. Herr, Somerville, MA (US); Jiun-Yih Kuan, Cambridge, MA (US)

(21) Appl. No.: 15/612,597

(22) Filed: Jun. 2, 2017

Related U.S. Application Data
(60) Provisional application No. 62/344,635, filed on Jun. 2, 2016.

(51) Int. Cl. A61H 3/00; A61F 2/66; F16H 19/00; A61F 2/50

(52) U.S. Cl. CPC A61H 3/00; F16H 19/005; A61F 2/6607; A61F 2002/503; A61F 2002/5038

(57) ABSTRACT

A mechanical transmission, tethered actuation system, an autonomous ankle exoskeleton design and method of their use employing a cable, pulleys and associated pulley housings to change angular transmission of linear force on the cable. The pulleys are linked by a ground link and the cable is threaded across and between the pulleys, whereby rotation of either of the pulleys in one direction causes rotation of the other pulley in the opposite direction. Independently of the pulleys, the pulley housings can freely rotate about associated pulleys, and a link between the pulley housings is provided, whereby rotation of one of the pulley housings in one direction causes rotation of the other pulley housing at an equivalent angle in the opposite direction, thereby enabling a change in transmission angle of linear force on the cable threaded across and between the pulleys and the associated pulley housing essentially without resistance. When pulleys have the same angular velocity ratio as that of the associated pulley housings, there is no cable slack since the net changes in length of the cable wrapping around two pulleys is zero.

### RELATED APPLICATION

This application claims the benefit of U.S. Provisional Application No. 62/344,635, filed on Jun. 2, 2016. The entire teachings of the above application are incorporated herein by reference.

### GOVERNMENT SUPPORT

This invention was made with government support under Grant No. NNX12AM16G from the National Aeronautics and Space Administration. The government has certain rights in the invention.

### BACKGROUND

Cables have long been regarded as one of the most flexible ways to transmit mechanical power and motion, especially, for long-distance actuation where the motor input is spatially separated from the output end effector. A non-exhaustive list of common cable driven devices include wearable robotic emulator devices for prosthetic, orthotic and exoskeletal devices; robotic therapy tools, robotic surgical tools, bicycle brakes, dental drills, hair shearing, and cranes. In order to transmit mechanical power and motion from an input to an output, cable housings or frames must be deployed to provide the required reaction forces to the actuation. Compared with other mechanical power transmissions, such as linkages and gears, cables are relatively lightweight and flexible but may suffer from friction losses and cable slackness, leading to poor control performance.

To achieve a high-performance cable transmission, typically engineers use a multiple-stage pulley system and a pretension mechanism, such as the devices taught in US 2007/0149328 A1 [1] and U.S. Pat. No. 7,736,254 B2 [2]. However, such devices are usually complex and bulky, limiting the size and flexibility of the systems. In order to avoid cable slackness, some devices, such as dental drills and bicycle brakes, make use of elastic bands and/or springs. This can introduce compliance to the systems, deteriorating the efficacy of the devices. Some high-performance cable transmission has been achieved by utilizing complicated pulley systems and pretension mechanisms, such as in a teleoperator system using a Whole-Arm Manipulation (WAM) robot [3], [4] and in laparoscopic surgery robots with multiple pulleys and pretension mechanisms [5], [6], [7]. However, cable housings or frames of these devices using multiple pulleys must be specifically designed and are usually bulky, limiting the size and flexibility of the systems, and inevitably leading to a difficulty in use in different forms.

As one type of flexible cable, Bowden cables are used to increase the flexibility of the transmission by deploying a hollow flexible outer cable conduit, which consists of an inner lining, a longitudinally incompressible layer, and a protective outer covering [8]. Typically, Bowden cables are commonly used in conjunction with aircraft control and bicycle brakes. They are now also frequently used with wearable devices, such as described in [9], [8], because they are lightweight and flexible, and because human movement is often unpredictable. Compared to Bowden cables, which are flexible, rigid mechanical transmission with limited degrees of freedom are usually intended to impede human motions by adding weights and/or restricting body motions. However, in order to provide large output power, the cable conduit has to be strong enough to provide required reaction forces, and consequently, the conduit becomes stiffer and heavier. When the cable is under tension, reaction forces on the conduit tend to straighten the cable conduit, causing external lateral impedance against the outside environment. Moreover, Bowden cables also often suffer from inefficiency and variations in cable tension due to bending of the cable housing and to friction losses [10]. An improved Bowden cable system has been designed to minimize friction resistance and to provide a better directional stability, as well as a much narrower and tension-free disposition in curves [11]. However, the friction losses are still significant when the cable is bending in a curvature due to changes of transmission angle between the input and output, and the forces between each cable segment of a conduit tend to lock the conduit in its current position.

Therefore, a need exists for a mechanical transmission and tethered actuation system that overcomes or minimizes the above-referenced problems.

### SUMMARY OF THE INVENTION

The invention generally is directed to a mechanical transmission, a tethered actuation system, a method of actuating an end effector, and an autonomous ankle exoskeleton design.

In one embodiment, the mechanical transmission includes a ground link having first and second pivots defining parallel axes of rotation, a first pulley rotatable about the first pivot, and a second pulley rotatable about the second pivot. A first pulley housing rotates about the first pivot in response to a change in transmission angle of linear force of a cable at the first pulley, the cable being threaded across and between the pulleys. A second pulley housing rotates about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of force across the mechanical transmission.

In another embodiment, a tethered actuation system of the invention includes an input mechanism, an output mechanism and a cable linking the input mechanism and the output mechanism. At least one mechanical transmission includes a ground link having first and second pivots that define parallel axes of rotation, a first pulley rotatable about the first pivot, and a second pulley rotatable about the second pivot. A first pulley housing rotates about the first pivot in response to a change in transmission angle of linear force of the cable at the first pulley, the cable being threaded across and between the pulleys. A second pulley housing rotates about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of force across the mechanical transmission. A first cable housing extends about the cable between the input mechanism and the at least one mechanical transmission. A second cable housing extends about the cable between the at least one mechanical transmission and the output mechanism.

In yet another embodiment, the invention is a method of actuating an end effector, comprising the step of actuating an input mechanism, whereby force is transmitted from the input mechanism to an output mechanism through a cable that extends across at least one mechanical transmission, the at least one mechanical transmission including a ground link having first and second pivots that define parallel axes of rotation A first pulley is rotatable about the first pivot, and a second pulley is rotatable about the second pivot. A first pulley housing rotates about the first pivot in response to a change in transmission angle of linear force of the cable at the first pulley, the cable being threaded across and between the pulleys. A second pulley housing rotates about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of force across the mechanical transmission.

In still another embodiment, the invention is an ankle exoskeleton system design, comprising an electric motor, an input mechanism, an output mechanism, a cable linking the input mechanism and the output mechanism, and at least one mechanical transmission, including a ground link having first and second pivots that define parallel axes of rotation, a first pulley rotatable about the first pivot, and a second pulley rotatable about the second pivot. A first pulley housing rotates about the first pivot in response to a change in transmission angle of linear force of the cable at the first pulley, the cable being threaded across and between the pulleys. A second pulley housing rotates about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of force across the mechanical transmission. A first cable housing extends between the input mechanism and the at least one mechanical transmission, and a second cable housing extends between the at least one mechanical transmission and the output mechanism.

In one embodiment, the invention is a wearable device, including a distal member wearable by an individual distal to a skeletal joint of the individual, a proximal member, a link between the distal member and the proximal member, and at least one mechanical transmission. The proximal member includes a tube, an actuator and a harness wearable by the individual proximal to the joint, wherein one or the other of the distal members and the proximal member includes an elastic crossing member. The elastic crossing member and the link span an axis about which the distal member rotates, from one to the other of the distal member or the proximal member, and whereby actuation of the link is translated into a force at the distal or proximal member that is normal a major longitudinal axis extending through the distal and proximal members. A cable is connected to the crossing member and extends from the crossing member to the actuator. The mechanical transmission is between at least one of: the distal member and the proximal member; and the actuator and the tube, the mechanical transmission including a ground link defining parallel first and second pivots, a first pulley rotatable about the first pivot, and a second pulley rotatable about the second pivot. A first pulley housing rotates about the first pivot in response to a change in transmission angle of linear force of the cable at the first pulley, the cable being threaded across and between the pulleys. A second pulley housing rotates about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of force across the mechanical transmission.

This invention includes many advantages. For example, the mechanical transmission, the tethered actuation system of the invention, and the method of the invention can efficiently transmit motion and mechanical power from an input device, such as a motor, to an output device, or end-effector, via a cable or its equivalent, such as a rope. With a high level of efficiency and minimal frictional forces in the transmission, angular transmission of linear force on the cable or rope can be effected without physical constraints on the location of the output relative to the input in three-dimensional space. Since the mechanical transmission significantly reduces friction resistance and significantly reduces cable slackness, independent of the location of the output relative to the input, it is highly backdrivable. Specifically, an embodiment of the mechanical transmission of the invention is compact, modular, lightweight, stiff, highly backdrivable and free to rotate in three-dimensional space with virtually zero backlash between the transmission's input and the output. Since the mechanical transmission of the invention is compact and modular, and since it can be used for both bidirectional and unidirectional actuation, it is useful for many applications. Moreover, the angular velocity ratio of two pulley housings need not be 1:1, and it can be programmable as a variable ratio by changing the shape or design of the coupling components. Such a design could be useful for some applications in which the tension of the cable should change when the transmission angle between the input and the output changes. When pulleys have the same angular velocity ratio as that of associated pulley housings, the force balance is still valid and there is no cable slack since the net changes in length of the cable wrapping around two pulleys is zero. It may also be beneficial to use more than two pairs of pulley housings and pulleys in the same transmission using the same principle. For instance, using multiple pairs of pulley housings and pulleys in one transmission eliminates the need of any cable housings.

Because the cable transmission is compact, modular, lightweight, stiff, highly backdrivable and free to rotate in three-dimensional space, it can easily be used as a general mechanical component for different applications, such as an emulator system for wearable devices, surgical robotics, therapy robotics, flexible dental drills, hair shearing and teleoperation.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a topology of one embodiment a mechanical transmission of the invention.

FIGS. 2A and 2B illustrate a topology of a mechanical transmission of FIG. 1 and motion from one position (FIG. 1) to a second position (FIG. 2A), wherein the transition angle has increased from 0° to 90°, and from the second position (FIG. 2A) to a third position (FIG. 2B), wherein the transmission angle has increased from 90° to 180°.

FIG. 3 is a perspective view of one embodiment of a mechanical transmission of the invention.

FIG. 4 is a side view of the mechanical transmission shown in FIG. 3.

FIG. 5 is a plan view of the mechanical transmission shown in FIG. 3 and FIG. 4.

FIG. 6 is an exploded view of the mechanical transmission shown in FIGS. 3-5.

FIG. 7 is a perspective view of a tethered actuator employing a mechanical transmission of the invention.

FIG. 8 is a block diagram of one embodiment of an example control system for use with a tethered actuation system of the invention, employing two mechanical transmissions of the invention that are connected in parallel in a Bowden cable system.

FIG. 9 is a perspective view of another embodiment of a mechanical transmission of the invention.

FIG. 10 is an exploded view of the mechanical transmission shown in FIG. 9.

FIG. 11 is a perspective view of one embodiment of a bidirectional mechanical transmission of the invention.

FIG. 12 is an exploded view of the mechanical transmission shown in FIG. 11, wherein two cables, or two portions of a single cable, cross each other at a centerline between respective pulleys.

FIG. 13 is a perspective view of one two-degrees-of-freedom mechanical transmission consisting of two unidirectional mechanical transmissions shown in FIGS. 10-11.

FIG. 14 is a perspective view of an autonomous ankle exoskeleton device employing a mechanical transmission of the invention.

FIG. 15 is a frontal view of an autonomous ankle exoskeleton device shown in FIG. 14.

FIG. 16 is a perspective view of one embodiment of a bidirectional mechanical transmission of the invention wherein two cables, or two portions of a single cable, are essentially parallel to each other at a centerline between respective pulleys.

### DETAILED DESCRIPTION OF THE INVENTION

Generally, the invention is directed to a mechanical transmission that can efficiently transmit motion and power from an input to an output via a cable/rope essentially without physical constraints on the direction angles between the output motion and mechanical interface motion in three-dimensional space.

The invention generally is directed to a mechanical transmission, a tethered actuation system, a method of actuating an end effector, and to an autonomous ankle exoskeleton design.

In one embodiment, the invention is a mechanical transmission that includes a ground link having first and second pivots that define parallel axes of rotation. A first pulley is rotatable about the first pivot, and a second pulley is rotatable of the second pivot. A cable is threaded across and between the pulleys, whereby rotation of either the first pulley or the second pulley in one direction causes rotation of the other pulley in the opposite direction. In addition, a first pulley housing is rotatable about the first pivot in response to a change in a transmission angle of linear force of the cable at the first pulley. A second pulley housing is rotatable about the second pivot in response to a change in transmission angle of linear force of the cable at the second pulley. A transmission link is located between the pulley housings, whereby rotation of one of the pulley housings in one direction causes rotation of the other pulley housing in the opposite direction thereby causing the first and second pulley housings to rotate about the first and second pivots, respectively, of the ground link in response to the change in transmission angle of force across the mechanical transmission.

In another embodiment, the mechanical transmission further includes an adapter fixed to the second pulley housing, wherein the adapter defines a first axis that is parallel to the axis of rotation of the first pivot, and a second axis transverse to the axis of rotation of the first pivot. A second ground link defines a third pivot and a fourth pivot, the third and fourth pivots define distinct axes of rotation parallel to the second axis. A third pulley is rotatable about the third pivot and the fourth pulley is rotatable about the fourth pivot. A third pulley housing rotates about the third pivot in response to a change in transmission angle of linear force of a cable threaded across and between the third and fourth pulleys. The third pulley housing is attached to the adapter. A fourth pulley housing rotates about the fourth pivot in response to a change in transmission angle of linear force of the cable at the fourth pulley. A transmission link is between the third and fourth pulley housings, whereby rotation of one of the third and fourth pulley housings in one direction causes rotation of the other of the third and fourth pulley housings in the opposite direction, thereby causing the third and fourth pulley housings to rotate about the third and fourth pivots, respectively, and about the second ground link, in response to a change in transmission angle of linear force across the third and fourth pivots.

In one embodiment, the mechanical transmission of the invention includes first and second pulleys that are of about equal diameter. In another embodiment, the first and second pulleys are of different diameters. The transmission link between pulley housings can be a pair of agonist and antagonist tendons wrapped in opposite directions about and between the housings. Alternatively, the link between the first and second pulley housings can include a pair of conjugated gears affixed to the pulley housings, wherein teeth of each gear are engaged with the teeth of the other gear, thereby causing rotation of one of the pulley housing in one direction to rotate the other pulley housing in the opposite direction. A cable housing can be coupled to each pulley housing and extends away from each respective pulley. A cable can extend within the housings and across and between the pulleys; and the cable can further extend through the cable housings. In one embodiment, each cable housing is rotatable about an axis coaxial to a major longitudinal axis of the cable through the associated cable housing. The transmission can further include a suspension handle at the ground link.

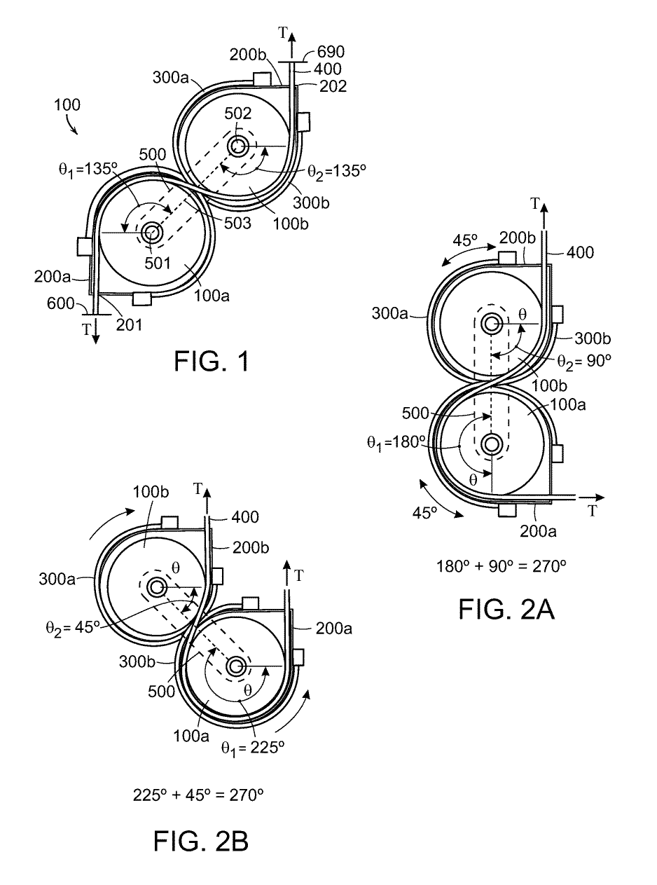

In one embodiment, shown in FIG. 1, the mechanical transmission 100 of the invention includes a pair of pulleys 100a, 100b, a pair of pulley housings 200a, 200b, a pair of agonist and antagonist tendons 300a, 300b, inner cable 400, and rotating arm 500. Rotating arm 500 operates as a ground link, and all motions are defined with respect to rotating arm 500 as a ground link. Preferably, rotating arm 500 is rigid, but could be elastic as well.

Pulley housings 200a, 200b and pulleys 100a, 100b rotate with respect to centers (pivots) 501, 502 defined by rotating arm 500. Pulley housings 200a, 200b, having the same pitch diameter (the diameter of the standard pitch circle), are coupled by agonist and antagonist tendons 300a, 300b so that the angular velocity ratio of pulley housings 200a, 200b is 1:1. Pulleys 100a, 100b, having the same pitch diameter, are free to rotate with respect to associated pulley housings 200a, 200b but are driven by inner cable 400. Inner cable 400 passes across and between pulleys 100a, 100b. From input end 201 of housing 200a, inner cable 400 wraps around one pulley 100a, and crosses a line 503 between centers 501, 502, between two pulleys 100a, 100b, and then wraps around the other pulley 100b in the opposite direction, going to output end 202 of housing 200b. Inner cable 400 can transmit motion and power from input end 201 to output end 202, driving pulleys 100a, 100b to rotate in opposite directions of rotation relative to each other. When cable 400 is pulled in the opposite direction, end 202 becomes the input end and end 201 becomes the output end. Since inner cable 400 is guided by pulleys 100a, 100b essentially without resistance or slippage, the transmission has a very low friction loss and is highly backdrivable.

Herein, the “transmission angle θₜᵣₐₙₛ” is defined as the angle difference between the input and the output, and its domain is −180°<θₜᵣₐₙₛ≦180°. It is zero when the input and output are collinear in the same direction and the sign of the transmission angle follows the right-hand rule by convention. The “wrap angle θ_{wrap}” is defined herein as the sum of the angles around pulleys 100a, 100b wrapped by inner rope 400, and thus it is always positive. The initial condition is defined as θₜᵣₐₙₛ=0°, and accordingly, θ is the angle displacement of the tangential point of the cable on the pulley with respect to that in the initial condition. As shown in FIGS. 2A-2B, when the transmission angle between the input and the output changes, it causes both pulley housings 200a, 200b to rotate with respect to their respective centers of rotation 501, 502 on line 503 (FIG. 1) at an equivalent angle θ but in opposite directions. Specifically, two pulley housings 200a, 200b must be driven simultaneously at an equivalent angle but in opposite directions of rotation due to the coupling of agonist and antagonist tendons 300a, 300b. As a result, the resultant change of the transmission angle is twice the angle change of one of the housings, namely, 2θ. However, the wrap angle of inner cable 400 around pulleys 100a, 100b is always constant since the changes of the transmission angle are compensated by the resultant change of the angles of both pulley housings 200a, 200b. The relationship between θₜᵣₐₙₛ and θ_{wrap} can be described by the following equations:

θ_{wrap}=θₒ−θ+θ=θₒ (1)

θₜᵣₐₙₛ=270°−θ_{wrap}+2·θ (2)

where θₒ is the preset wrap angle when running inner cable 400 through transmission 100 the first time, so it can be adjusted by changing the initial transmission angle. For example, as can be seen in FIG. 1, the preset wrap angle θₒ is 270°, while the component wrap angles, are θ₁=135° and θ₂=135° C., where θ₁+θ₂=270°. Whatever the change in transmission angle, the wrap angle remains constant, implying zero slackness of cable 400; to wit, there is effectively no backlash between the input and the output of cable 400 due to change in the transmission angle of linear force T on cable 400. FIGS. 2A-2B illustrate a topology of a mechanical (or free rolling cable) transmission of FIG. 1 in motion from one position (FIG. 1) to a second position (FIG. 2A) where the transmission angle has increased from 0° to 90°, and from the second position (FIG. 2A) to a third position (FIG. 2B), where the transmission angle has increased from 90° to 180°. As can be seen in FIG. 2A, the component wrap angles, θ₁ and θ₂, have changed to 225° and 45°, respectively, and again total 270°.

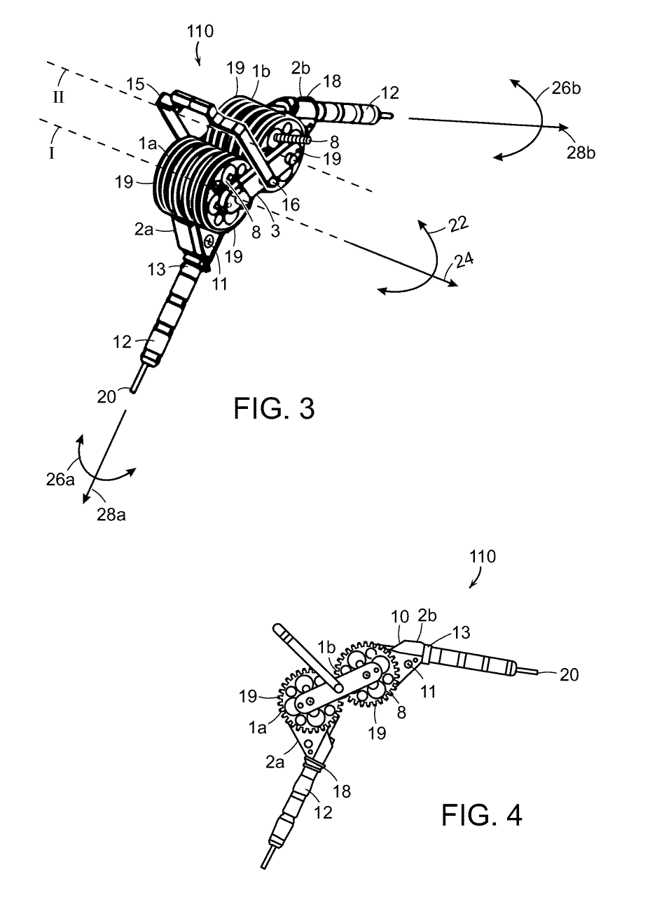

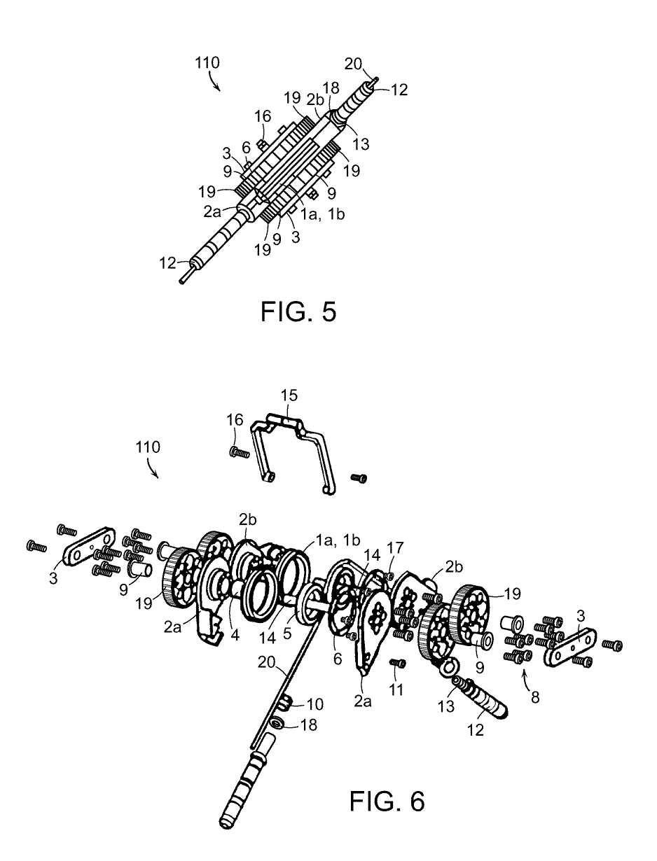

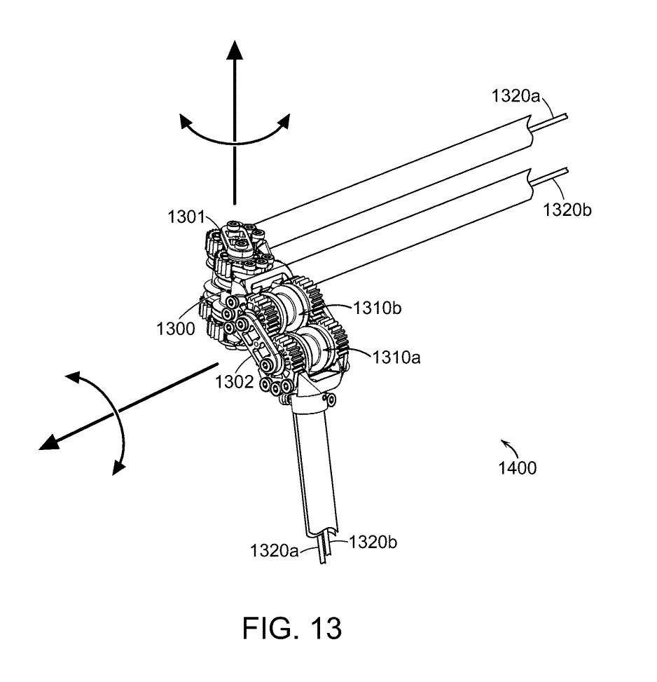

When inner cable 400 is under tension T, it tends to push two pulleys 100a, 100b away from each other, rotating the whole transmission mechanism. However, agonist and antagonist tendons 300a, 300b and external cable housings contribute to the resultant reaction force on each housing 200a and 200b, balancing the resultant force on the associated pulleys 100a and 100b, respectively. Therefore, the total sum of forces, except the weight, on rotating arm 500, is always zero. Accordingly, even if inner cable 400 is under great tension, transmission mechanism 100 is free to rotate and thus free to translate. Agonist and antagonist tendons 300a, 300b can be substituted with alternative coupling components that keep a 1:1 angular velocity ratio between two pulley housings 200a, 200b, such as a pair of gears, belts, linkages, etc. (See, e.g., gears 19 of FIGS. 3-6). Moreover, the angular velocity ratio between two pulley housings 200a, 200b can be other than 1:1, such as that the radius of pulley housing 200a could be 10 mm and the radius of pulley housing 200b could be 100 mm, so the angular velocity ratio between pulley housings 200a, 200b would be 10:1. When pulleys 100a, 100b also have the same angular velocity ratio 10:1 as that of pulley housings 200a, 200b, the force balance is still valid and there is no cable slack since the net changes in length of the cable wrapping around two pulleys is zero. In one embodiment, the angular velocity ratio of pulley housing 200a, 200b, can be programmable as a variable ratio by changing the shape or design of the coupling components, such as using a pair of elliptical gears. It is also to be understood that the number of pulley housings and pulleys employed in one transmission can be more than two, such as embodiment 1400 shown in FIG. 13, which has one common pulley housing, three independent pulley housings and four pulleys.

Moreover, because of the flexible nature of cable 400, the input end and the output end of the transmission can rotate with respect to the housing 200a, 200b in directions orthogonal to the centers of the rotation of pulley housings 200a, 200b. For example, the transmission can rotate about a major longitudinal axis of cable 400 extending from a point of contact with pulley 100a orthogonally to plane 600 (FIG. 1) and about a major longitudinal axis of cable 400 extending from a point of contact with pulley 100b orthogonally to plane 690 (FIG. 1). Accordingly, the transmission cannot only efficiently transmit motion and power, but also be free to rotate in three-dimensional space.

FIGS. 3, 4, 5 and 6 represent different views of one embodiment of a mechanical (or free rolling cable) transmission of the invention. As shown therein, mechanical transmission 110 includes a pair of pulleys 1a, 1b, a pair of pulley housings 2a, 2b, two pairs of conjugated gears 19, and a pair of rotating arms 3. Pivots at rotating arms 3 define axes I, II, about which pulleys 1a, 1b, and pulley housings 2a, 2b, rotate, respectively. Axes I, II are parallel to each other. In the example shown, each pulley housing is formed of two half parts. The half parts of pulley housing 2a and the half parts of pulley housing 2b are affixed by screws 8 and 11, respectively (see FIG. 6). Pulley housings 2a, 2b have the same pitch diameter while pulleys 1a, 1b have the same pitch diameter, but are smaller or equal to that of pulley housings 2a, 2b to avoid interference. Aforementioned agonist and antagonist tendons 300a, 300b are replaced by two pairs of conjugated gears 19.

As illustrated in FIG. 6, two ball bearings 5, are incorporated into pulleys 1a, 1b and secured by two bearing caps 6 and screws 17, so that the two pulleys are free to rotate with respect to the associated pulley housings 2a, 2b. Two needle bearings 4 and four thrust bushings 9 are incorporated into pulley housings 2a, 2b. Each of two axles 14 running through one needle bearing 4 and two thrust bushings 9 is fixed to the two rotating arms 3 by screws, so that pulley housings 2a, 2b are free to rotate with respect to rotating arms 3 positioned on either side of the housings. Therefore, pulley housings 2a, 2b and pulleys 1a, 1b can independently rotate about two axles 14 with respect to rotating arms 3 with little friction. Two pairs of gears 19, having the same pitch diameter, are affixed to pulley housings 2a, 2b by screws 8, so that the angular velocity ratio of two housings 2a, 2b is 1:1.

Cable housings 12 are attached to pulley housings 2a, 2b by threaded connectors 13 and nuts 10. Thrust bearings 18 between cable housings 12 and pulley housing 2a, 2b enable cable housings 12 to rotate with respect to the pulley housings about an axis orthogonal to the centers of rotation of the pulley housings. Accordingly, transmission 110 is free to rotate in three-dimensional space. Arrow 22 in FIG. 3 illustrates rotation of pulley housings 2a, 2b about axis 24 that passes through the center of rotation of pulley housing 2a. As illustrated by arrows 26a, 26b in FIG. 3, each cable housings 12 can rotate about axes 28a, 28b that are orthogonal to axis 24. In the example shown, axes 28a, 28b and two major longitudinal axes of cable 400 running through two cable housings 12 are coaxial, respectively.

Pulleys 1a, 1b are free to rotate with respect to the associated housings 2a, 2b but are driven by inner cable 20. From the input end through one cable housing 12, inner cable 20 wraps around one of two pulleys 1a, 1b, crosses the line of the centers of the two pulleys, and then wraps around the other pulley 1 in the opposite direction, going to the output end through the other cable housing 12. Inner cable 20 can transmit motion and power from the input to the output, driving the pulleys to rotate in the opposite directions of rotation. Since inner cable 20 is guided by the pulleys the transmission has a very low friction loss and thus is highly backdrivable.

The total sum of forces, disregarding mechanism weight, on rotating arms 3 is always zero; accordingly, even if inner cable 20 is under great tension, the transmission is free to rotate. To lift the transmission, suspension handle 15 is bolted to rotating arms 3 by shoulder screws 16, so that suspension handle 15, supported by a point force (such as tension force on a rope), can provide the force against the weight of the transmission while allowing the transmission to rotate in three-dimensional space.

Embodiments of the method of actuating and actuator system that are described herein can be used with the system and devices described in U.S. Published Application No.: 2013/0158444, entitled “A Robotic System for Simulating a Wearable Device and Method of Use,” by Herr et al., now U.S. Pat. No. 9,498,401, the relevant teachings of which are incorporated herein by reference.

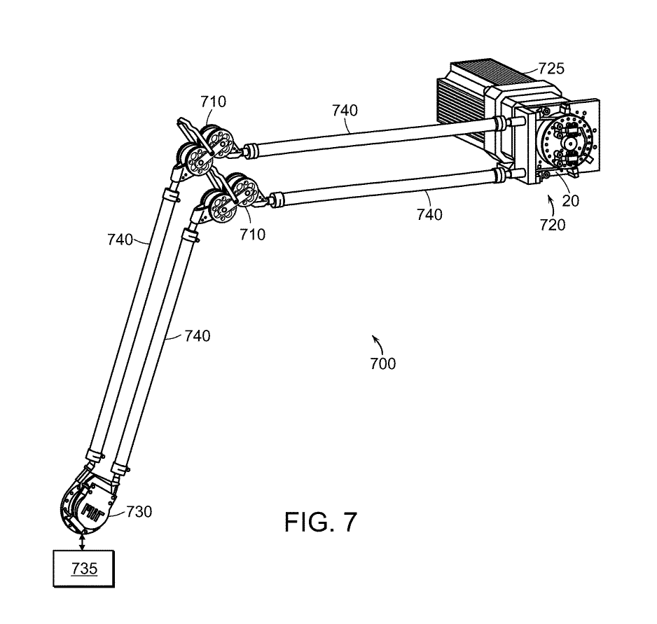

In another embodiment, shown in FIG. 7, a tethered actuation system of the invention includes an input mechanism, an output mechanism, and a cable linking the input mechanism and the output mechanism. The at least one mechanical transmission includes a ground link having first and second pivots that define parallel axes of rotation. A first pulley is rotatable about the first pivot, and a second pulley is rotatable of the second pivot. A cable is threaded across and between the pulleys, whereby rotation of either the first pulley or the second pulley in one direction causes rotation of the other pulley in the opposite direction. In addition, a first pulley housing is rotatable about the first pivot in response to a change in a transmission angle of linear force of the cable at the second pulley. A second pulley housing is rotatable about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link in response to the change in transmission angle of force across the mechanical transmission. A first cable housing extends between the input mechanism and the mechanical transmission. A second cable housing extends between the mechanical transmission and the output mechanism.

More specifically, FIG. 7 illustrates one embodiment of a tethered actuator system 700 that employs a mechanical transmission of the invention for one-degree-of-freedom tethered actuation. Tethered actuator is bidirectional and the system includes two free rolling cable transmissions 710, input mechanism 720, output mechanism 730, and cable housings 740. Input mechanism 720 transmits power to output mechanism 730 by movement of inner cable 20, 400 (FIGS. 1-2B) relative to cable housings 740 and transmissions 710. A motor 725 at input mechanism 720 provides actuation to move cable 20. Output mechanism 730 is coupled to end-effector 735 and can be configured to move, or otherwise actuate, the end-effector.

In one particular embodiment, the tethered actuation system of the invention further includes a control system that is in communication with the input mechanism and the output mechanism. The control system includes a host computer that includes a user interface, a master controller in communication with the host computer and that provides real-time control and sensor fusion. A local servo controller is in communication with the master controller and input mechanism, the local servo controller controlling the input mechanism. Sensors transmit measurements of output states from the output mechanism, and input/output modules convert signals from the sensors and transmit the converted signals to the master controller, whereby a torque command is produced and communicated to the input mechanism using measured feedback states from the sensors.

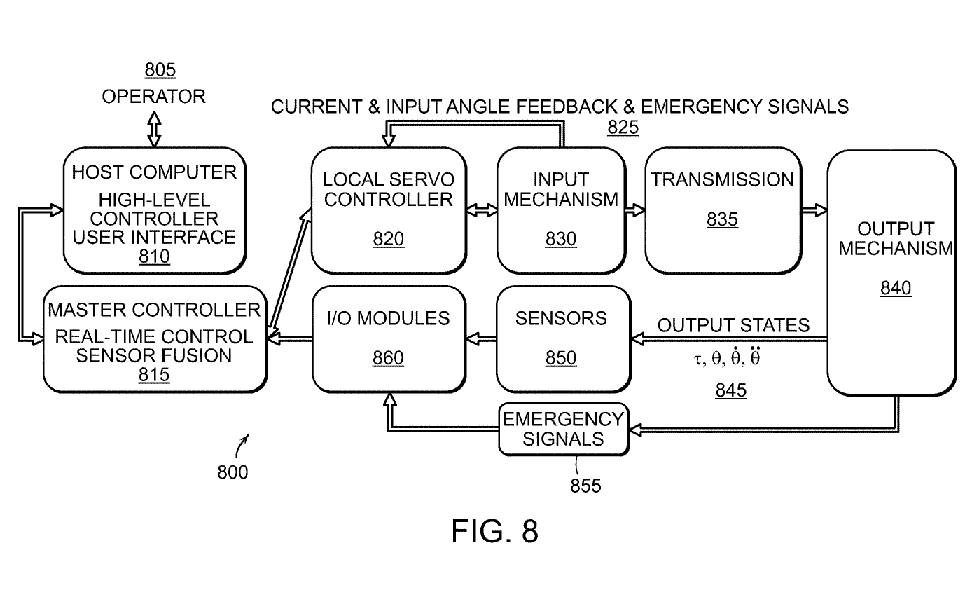

A method of actuating an end-effector, such as by use of a control system, as shown in FIG. 8, includes the step of actuating an input mechanism, whereby force is transmitted from the input mechanism to an output mechanism through a cable that extends across at least one mechanical transmission, the at least one mechanical transmission including a ground link that defines a plurality of pivots, a first pulley rotatable about one of the pivots, and a second pulley rotatable about another of the pivots and linked to the first pulley by the ground link having first and second pivots that define parallel axes of rotation. A first pulley is rotatable about the first pivot, and a second pulley is rotatable of the second pivot. A cable is threaded across and between the pulleys, whereby rotation of either the first pulley or the second pulley in one direction causes rotation of the other pulley in the opposite direction. In addition, a first pulley housing is rotatable about the first pivot in response to a change in a transmission angle of linear force of the cable at the second pulley. A second pulley housing is rotatable about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link in response to the change in transmission angle of force across the mechanical transmission. A first cable housing extends between the input mechanism and the mechanical transmission. A second cable housing extends between the mechanical transmission and the output mechanism.

FIG. 8 is a schematic representation of one embodiment of a general control system 800 of the invention for a system that employs a mechanical transmission of the invention. In order to ensure system performance and safety, the user interface and high-level control algorithms, e.g. biophysical control and virtual model control, are implemented in a host computer 810; real-time control, e.g. virtual model control, impedance control, output torque control, output position control and sensor fusion, (utilizing multiple sensors to assist the accuracy of the feedback system), are implemented in a standalone master controller 815; real-time servo control, e.g. current control, input position control, are implemented in a local servo controller 820. An input/output (I/O) system 860, which includes one or more I/O modules, is connected to master controller 815. I/O system 860 has multiple digital inputs and multiple digital outputs, which can be used for gathering, via sensor 850, output states 845, such as encoder feedback signals, torque feedback signals, etc. Using these sensory data, master controller 815 can send torque commands to local servo controller 820, to thereby enforce desired output performance.

As illustrated in FIG. 8, local servo controller 820 is coupled to input mechanism 830 and receives current and input angle feedback 825 from the input mechanism. In addition, input mechanism 830 provides emergency signals to local servo controller 820, which can be used to inform the system that the output end is approaching the limitation of the range of motion, and thus the system can stop rapidly to avoid a dangerous event. Similarly, output mechanism 840 provides emergency signals 855 to I/O system 860 (I/O modules). The local servo controller controls input mechanism 830, which is connected to the output mechanism 840 via the transmission 835.

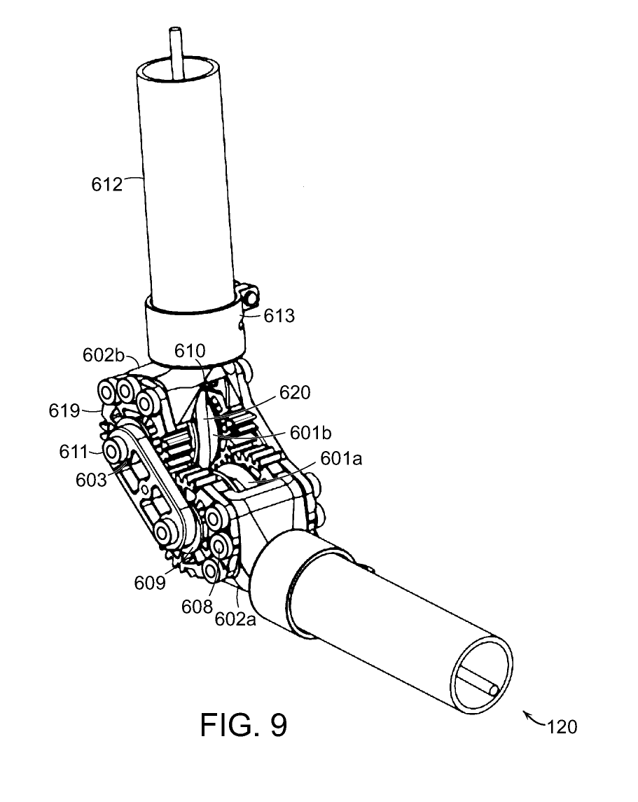

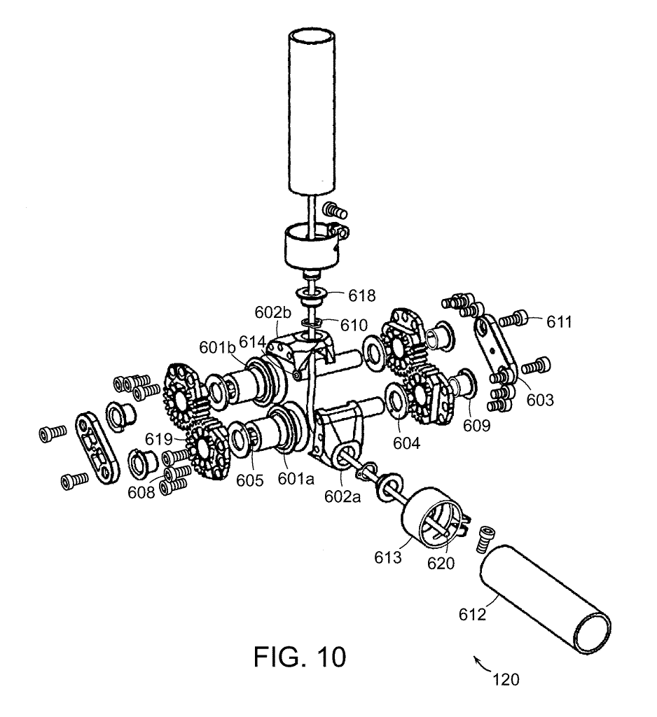

FIGS. 9 and 10 represent different views of another possible embodiment of a mechanical (or free rolling cable) transmission of the invention. As shown therein, mechanical transmission 120 includes a pair of pulleys 601a, 601b, a pair of pulley housings 602a, 602b, two pairs of gears 619, and a pair of rotating arms 603. Pulley housings 602a, 602b have the same pitch diameter while pulleys 601a, 601b have the same pitch diameter, but are smaller or equal to that of pulley housings 602a, 602b to avoid interference. Aforementioned agonist and antagonist tendons 300a, 300b are replaced by two pairs of conjugated gears 19.

As illustrated in FIG. 10, two needle bearings 605, are incorporated into pulleys 601a, 601b, and sandwiched between two thrust washers 604, so that the two pulleys 601a, 601b can rotate with respect to the associated pulley housings 602a, 602b. Each of two axles 614 running through one needle bearing 605, two thrust washers 604, and two thrust bushings 609 is fixed to the two rotating arms 603 by screws 611, so that pulley housings 602a, 602b are free to rotate with respect to rotating arms 603 positioned on either side of the housings. Therefore, pulley housings 602a, 602b and pulleys 601a, 601b can independently rotate about two axles 614 with respect to rotating arms 603 with little friction. Two pairs of conjugated gears 619, having the same pitch diameter, are affixed to pulley housings 602a, 602b by screws 608, so that the angular velocity ratio of two housings 602a, 602b is 1:1.

Cable housings 612 are attached to pulley housings 602a, 602b by adapters 613 and C-clips 610. Thrust bushings 618 between cable housings 612 and pulley housing 602a, 602benable cable housings 612 to rotate with respect to the pulley housings 602a, 602b about an axis orthogonal to the centers of rotation of the pulley housings. Accordingly, transmission 120 is free to rotate in three-dimensional space, in the same way as the first embodiment shown in FIG. 3.

Pulleys 601a, 601b are free to rotate with respect to the associated pulley housings 602a, 602b but are driven by inner cable 620. From the input end through one cable housing 612, inner cable 620 wraps around one of two pulleys 601a, 601b, crosses the line of the centers of pulleys 601a, 601b, and then wraps around the other pulley 601 in the opposite direction, going to the output end through the other cable housing 612. Inner cable 620 can transmit motion and power from the input to the output, driving the pulleys 601a, 601b to rotate in the opposite directions.

Embodiment 120 has fewer mechanical components and less weight than that of embodiment 110, so that no additional support structure may be needed.

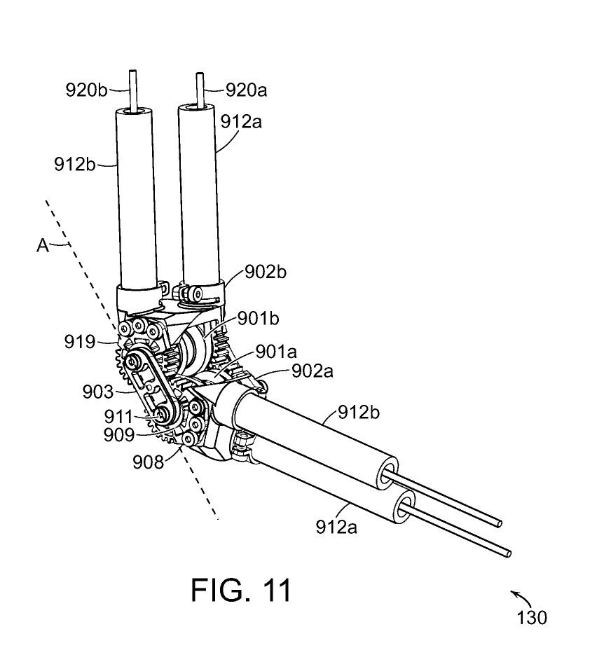

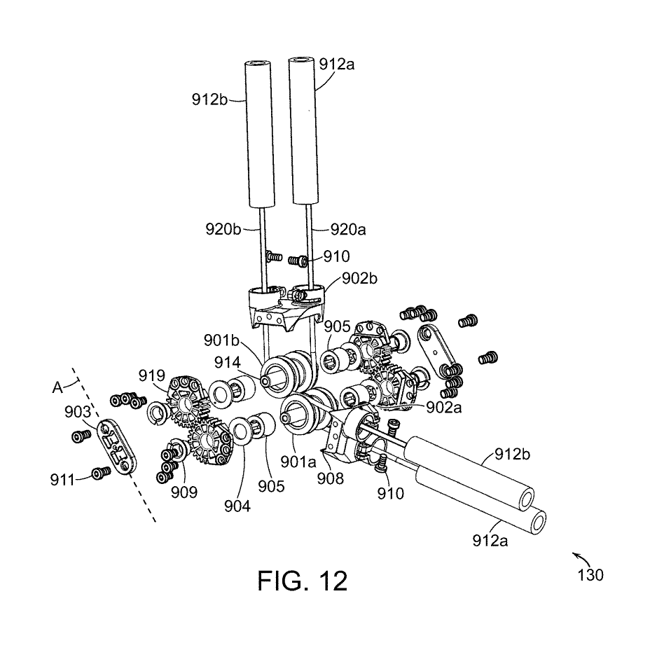

FIGS. 11 and 12 show one possible embodiment of a bidirectional mechanical transmission 130 of the invention. As shown in FIGS. 11 and 12, a first length of cable and the second length of cable extend across and between the third fourth pulleys, respectively, and cross each other at a centerline A between the axes of rotation of the first and second pulleys, and the third and fourth pulleys, respectively. In one embodiment of the invention, where the two lengths of cable are both part of a common cable, the first and second pulleys each include two grooves. In an alternative embodiment of the invention the the two lengths of cable are two cables that operate independently. In this alternative embodiment (not shown), a length of cable is a first length of cable, and the mechanical transmission of the invention further includes a third pulley rotatable about the first pivot and a fourth pulley rotatable about the second pivot, whereby a second length of cable, such that the first and second lengths of cable are two lengths of the same cable or two lengths of different cables, extend across and between the third and fourth pulleys. The first and third pulleys, rotate independently, and the second and fourth pulleys rotate independently.

More specifically, in one embodiment, mechanical transmission 130 includes a pair of pulleys 901a, 901b, a pair of pulley housings 902a, 902b, two pairs of conjugated gears 919, and a pair of rotating arms 903. Pulley housings 902a, 902b have the same pitch diameter while pulleys 901a, 901b have the same pitch diameter, but are smaller or equal to that of pulley housings 902a, 902b to avoid interference.

Mechanical transmission 130 shares a similar design to that of transmission 120 (FIGS. 9-11), to wit, two rotating arms 903 are fixed to two axles 914 by screws 911, and pulleys 901a, 901b and pulley housings 902a, 902b can rotate with respect to rotating arms 903 positioned on either side of the pulley housings. Each of two axles 914, running through one needle bearing 905, two thrust washers 904 and two thrust bushings, 909, are fixed to the two rotating arms 903 by screws 611. Two pairs of conjugated gears 619, having the same pitch diameter, are affixed to pulley housings 902a, 902b by screws 908, forcing the two pulley housings to rotate in the 1:1 angular velocity ratio. However, pulley housings 902a, 902b have built-in adapters to clamp cable housings 912a, 912b by screws 910. As a result, cable housings 912a, 912b are fixed to the pulley housings.

Moreover, both pulleys 901a, 901b have two grooves on each side to guide two inner cables 920a, 920b. Pulleys 901a, 901b are free to rotate with respect to the associated housings 902a, 902b but are simultaneously driven by both inner cables 920a, 920b on each side of the pulleys. From the input end through cable housing 912a, inner cable 920a wraps around one of two pulleys 901a, 901b, crosses the line of the centers of the two pulleys, and then wraps around the other of the two pulleys 901a, 901b in the opposite direction, going to the output end through the other cable housing 912a. Conversely, from the input end through cable housing 912b, inner cable 920b wraps around one of the two pulleys 901a, 901b, crosses the line of the centers of the two pulleys (centerline A in FIG. 12), and then wraps around the other of the pulleys 901a and 901b in the opposite direction, going to the output end through the other cable housing 912b. Consequently, inner cable 920b and inner cable 920a wrap around the same pulleys in the opposite directions of rotation. As a result, inner cables 920a, 920b can transmit bidirectional motion and power from the input to the output. Accordingly, transmission 130 is only free to rotate in one direction, unlike transmissions 110, 120 in the aforementioned embodiments 110, 120. However, one can add more degrees of freedom by adding extra adapters to connect any two consecutive transmissions. As shown in FIG. 13, with an adapter 1300 coupling two transmissions 1301 and 1302, transmission 1400 is free to rotate in two directions. Two inner cables 1320a, 1320b should be actuated in an agonist-antagonist way so there is little or no sliding between pulleys 1310a, 1310b and inner cables 1320a, 1320b, such as when being driven by a rotational joint. Arrows in FIG. 13 illustrates two centers of rotation of the embodiment.

In still another embodiment, the invention is an ankle exoskeleton system design, comprising an electric motor, an input mechanism, an output mechanism, a cable linking the input mechanism and the output mechanism, and at least one mechanical transmission, including a ground link having first and second pivots that define parallel axes of rotation, a first pulley rotatable about the first pivot, and a second pulley rotatable about the second pivot. A first pulley housing rotates about the first pivot in response to a change in transmission angle of linear force of the cable at the first pulley, the cable being threaded across and between the pulleys. A second pulley housing rotates about the second pivot in response to a change in transmission angle of linear force of the cable at the second pulley. A transmission link is located between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of force across the mechanical transmission. A first cable housing extends between the input mechanism and the at least one mechanical transmission, and a second cable housing extends between the at least one mechanical transmission and the output mechanism.

In one embodiment, the ankle exoskeleton system includes a first mechanical transmission and a second mechanical transmission connected in series. Another embodiment of an ankle exoskeleton system design of the invention includes a harness to which the first and second mechanical transmissions are connected, wherein the first mechanical transmission is fixed proximate to a human hip joint and the second mechanical transmission is fixed proximate to a knee of a human subject.

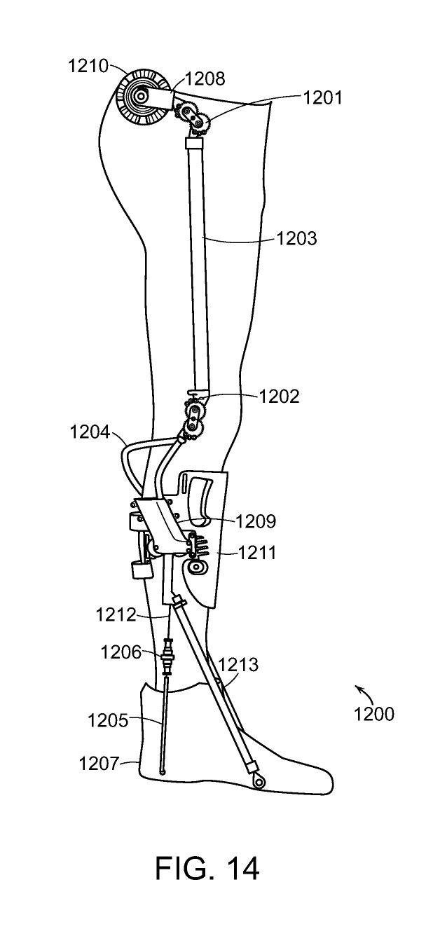

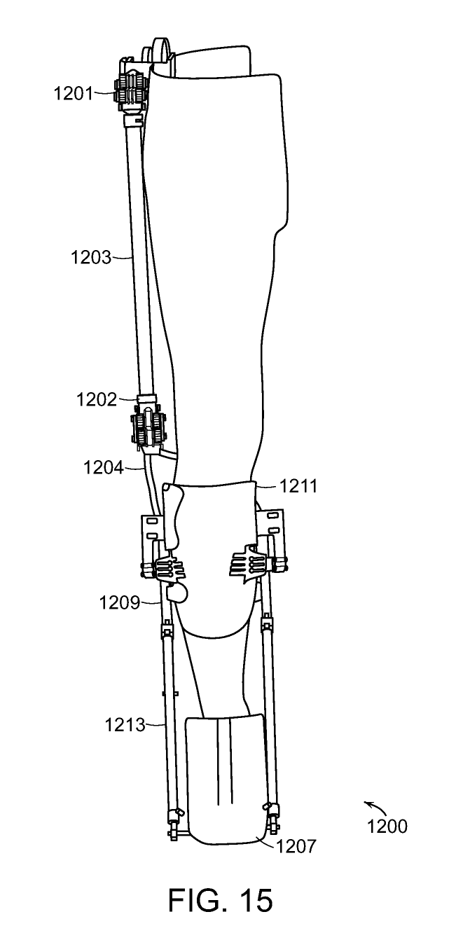

In another embodiment, shown in FIGS. 14 and 15, the invention is directed to a wearable device that includes a distal member 1207 wearable by an individual distal to a skeletal joint of the individual. A proximal member includes a tube 1203, an actuator 1210 and a harness 1214 wearable by the individual proximal to the joint, wherein one or the other of the distal member and the proximal member includes an elastic crossing member 1205. Links 1209, 1213 extend between the distal member and the proximal member, wherein the elastic crossing member and the link expand an axis about which the distal member rotates, from one to the other of the distal member or the proximal member. Actuation of the link is translated to a force at the distal or proximal member that is normal to a major longitudinal axis extending through the distal and proximal members. A cable is connected to the crossing member and extends from the crossing member to the actuator. At least one mechanical transmission is located between at least one of: the distal member and the proximal member; and the actuator and the tube. The mechanical transmission includes a ground link having first and second pivots that define parallel axes of rotation, a first pulley rotatable about the first pivot, a second pulley rotatable about the second pivot, a first pulley housing that rotates about the first pivot in response to a change in transmission angle of linear force at the first pulley of a cable threaded across and between the pulleys, a second pulley housing that rotates about the second pivot in response to a change of transmission angle of linear force at the second pulley of the cable, and a transmission link between the pulley housings. Rotation of one of the pulley housings in one direction causes rotation of the other pulley housing in the opposite direction, thereby causing the first and second pulley housings to rotate about the first and second pivots, respectively, of the ground link, in response to a change in angular transmission angle of linear force across the mechanical transmission.

As can be seen in FIGS. 14 and 15, the link includes strut 1219, wherein the strut extends from the proximal member to the distal member. The strut is constrained at the proximal member normally and laterally to a major longitudinal axis of the proximal member extending from the proximal member to the distal member. The strut is not restricted along the major longitudinal axis of the crossing member. The link includes at least one roller at the proximal member that constrains the strut normally and laterally. As shown, the link includes a pair of rollers in opposition to each other, wherein the strut is normally constrained between the pair of rollers. As also shown in FIGS. 14 and 15, the strut is curved at the pair of rollers 1218, whereby shear force between the strut and the pair of rollers during rotation of the distal member about the axis spanned by the crossing member and the strut is less than it would be if the strut were straight at the pair of rollers. Strut includes a guide tube at the pair of rollers, wherein the crossing member extends through the guide tube. The wearable device includes a pair of cross members and a pair of struts. Struts are essentially straight between the rollers and the distal member. Upon actuation, at least one of the struts deflects during eversion and inversion of the human foot secured to the distal member and a human calf secured to the proximal member. In an embodiment, the struts are rigid. In other embodiments, not shown, the struts are curved, whereby the struts operate as a series of springs during a normal walking cycle of a human foot secured to the distal member and a human calve secured to the proximal member. An example of such an embodiment is described in U.S. patent application Ser. No. 14/572,499, “Optimal Design of a Lower Limb Exoskeleton or Orthosis,” filed on Dec. 16, 2014 and published as US 2015/0209214 A1 on Jul. 30, 2015, the teachings of which are incorporated herein in their entirety. In still another embodiment, the link further includes a motor actuator assembly attached to a proximal end of the pair of crossing members, whereby actuation of the link will cause retraction of the crossing members, which causes rotation of the distal member and plantar flexion of a human foot secured to the distal member about a human ankle joint. As shown, the pair of crossing members is fixed to proximal end of the distal member.

More specifically, as shown in FIGS. 14 and 15, as one of applications of the embodiment, the configuration of transmissions for an autonomous ankle exoskeleton 1200 is proposed. The proposed invention mainly comprises an electric motor 1210, a motor mount 1208, two unidirectional transmission modules 1201, 1202, a long carbon fiber tube 1203, flexible conduit components 1204, and ankle end-effector 1290 worn by a wearer. Ankle end-effector 1290 mainly consists of a shank guard component 1211, a strut 1219, a pair of rollers 1218, guide tubes 1213, a force sensor 1206, an inner cable 1212, an elastic cable 1205, and an output shoe 1207. Strut 1219, rollers 1218, and guide tubes 1213 can be regarded as an input mechanism 1209 of ankle end-effector 1290. Motor 1210 is affixed to motor mount 1208 that is attached to harness 1214 around the waist of the wearer. Inner cable 1212 connects to motor 1210, running through transmission 1201, tube 1203, transmission 1202, flexible conduits 1204, strut 1209, and is affixed to proximal end of force sensor 1206. The distal end of force sensor 1206 is affixed to one end of elastic cable 1205, the other end of which is affixed to the ankle portion of shoe 1207. Shank guard component 1211 is mounted on the anterior shank of the wearer. Transmission 1201 is located next to the hip joint, allowing free abduction, adduction, rotation motions of the hip, and transmission 1202 is located next to the knee joint, allowing free flexion and extension motions of the knee. Flexible conduits 1204 are used to compensate small differences in motions between the transmission and the wear. When motor 1210 pulls inner cable 1212, the force is transmitted from motor 1210 through input mechanism 1209 to shoe 1207 while guide tubes 1213 provides the required reaction force also contributing the output force on 1207. The output force can be measured directly by force sensor 1206 or indirectly by measuring the extension of elastic cable 1205, since elastic cable 1205 serve as an artificial soleus that helps store the energy during walking or running. The other details of the ankle exoskeleton end-effector design can be found in [12]. It is to be understood that the proposed configuration of transmissions can be used with any cable-driven ankle end-effector.

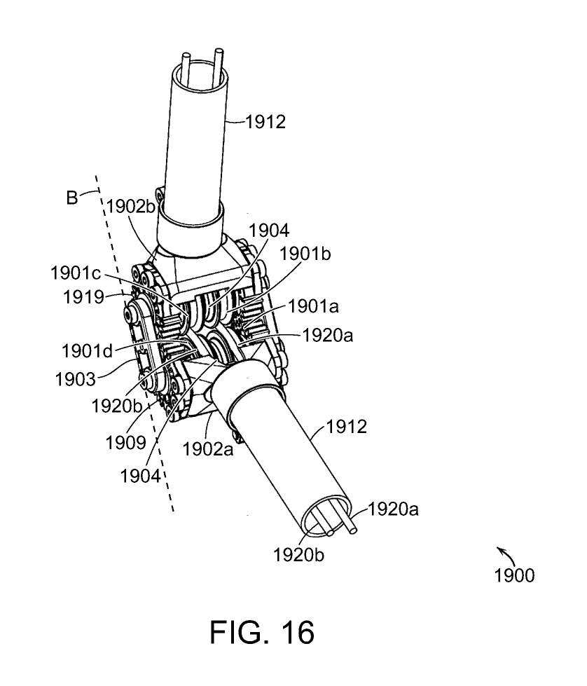

FIG. 16 shows another possible embodiment of a bidirectional mechanical transmission of the invention. As shown therein, the mechanical transmission of the invention further includes a third pulley rotatable about the first pivot and a fourth pulley rotatable about the second pivot. First and second lengths of cable are two lengths of the same cable or two lengths of different cables. The first length of cable extends across and between the first and second pulleys, and the second length of cable extends across and between the third and fourth pulleys respectively. Between the respective pulleys (the first and second pulley for the first cable, and the third and fourth pulley for the second cable) the first and second lengths of cable are essentially parallel to each other at centerline B between the axes of rotation of the first and second pulleys, and the third and fourth pulleys, respectively. Where the two lengths of the cable are two lengths of the same cable, the first and third pulleys rotate independently, and the second and fourth pulleys rotate independently. Where the two lengths of cable are two lengths of different cables, then the first and third pulleys can rotate independently, or be rotationally locked, and the second and fourth pulleys can rotate independently, or be rotationally locked, depending on the device in which they are employed.

As shown in FIG. 16, mechanical transmission 1900 includes two pairs of pulleys 1901a, 1901b, 1901c, 1901d, and a pair of pulley housings 1902a, 1902b, two pairs of conjugated gears 1919, 1909, and a pair of rotating arms 1903. Pulley housings 1902a, 1902b have the same pitch diameter while pulleys 1901a, 1901b, 1901c, 1901d have the same pitch diameter, but are smaller or equal to that of pulley housings 1902a, 1902b to avoid interference. Two thrust washers 1904 are sandwiched between pulleys 1901a, 1901b, and pulleys 1901c, 1901d, respectively, to allow the independent rotation between any two adjacent pulleys.

Mechanical transmission 1900 shares a similar design to that of embodiment 130 (FIGS. 11-12), except that, through tubes 1912, inner cable 1920a and inner cable 1920b extend across and between pulleys 1901a, 1901b and pulleys 1901c, 1901d, respectively, in the same direction, whereby inner cables 1920a, 1920b are essentially parallel to each other at the centerline (centerline B in FIG. 16) between the first and second pulleys 1901a, 1901b, and the third and fourth pulleys 1901d, 1901c, respectively.

### REFERENCES

[1] W. T. Townsend, “Automatic pretensioning mechanism for tension element drives,” US 2007/0149328 A1, 2007.
[2] B. M. Schena, “Compact cable tension tender device,” U.S. Pat. No. 7,736,254 B2, 2010.
[3] W. T. Townsend and J. a. Guertin, “Teleoperator slave—WAM design methodology,” Ind. Robot An Int. J., vol. 26, no. 3, pp. 167-177, 1999.
[4] J. K. Salisbury and W. T. Townsend, “Compact Cable Transmission with Cable Differential,” USO05207114A, 1993.
[5] S. P. Buerger, “Stable, high-force, low-impedance robotic actuators for human-interactive machines,” Massachusetts Institute of Technology, 2006.
[6] C. Y. Kim, M. C. Lee, R. B. Wicker, and S. M. Yoon, “Dynamic modeling of coupled tendon-driven system for surgical robot instrument,” Int. J. Precis. Eng. Manuf., vol. 15, no. 10, pp. 2077-2084, 2014.
[7] “Bowden cable,” Wikipedia. [Online]. Available at: https://en.wikipedia.org/wiki/Bowden_cable. (Downloaded Jun. 1, 2017)
[8] J. Kuan, K. A. Pasch, and H. M. Herr, “Design of a Knee Joint Mechanism that Adapts to Individual Physiology,” 36th Annu. Int. Conf. IEEE Eng. Med. Biol. Soc., pp. 2061-2064, 2014.
[9] J. F. Veneman, “A Series Elastic- and Bowden-Cable-Based Actuation System for Use as Torque Actuator in Exoskeleton-Type Robots,” Int. J. Rob. Res., vol. 25, no. 3, pp. 261-281, 2006.
[10] P. Letier, a Schiele, M. Avraam, M. Horodinca, and A. Preumont, “Bowden Cable Actuator for Torque-Feedback in Haptic Applications,” Eurohaptics, 2006.
[11] N. Noetzold, “Pull cable system,” U.S. Pat. No. 6,606,921 B2, 2003.
[12] Herr, Hugh M., et al. “Optimal design of a lower limb exoskeleton or orthosis.” U.S. patent application Ser. No. 14/572,499, 2014.

The relevant teachings of all references cited herein are incorporated herein in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A mechanical transmission, comprising:
a) a ground link having first and second pivots that define parallel axes of rotation;
b) a first pulley rotatable about the first pivot;
c) a second pulley rotatable about the second pivot;
d) a first pulley housing that rotates about the first pivot in response to a change in transmission angle of linear force of a cable at the first pulley, the cable being threaded across and between the pulleys;
e) a second pulley housing that rotates about the second pivot in response to a change in transmission angle of linear force of the cable at the second pulley; and
f) a transmission link between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of linear force across the mechanical transmission.

2. The transmission of claim 1, wherein the first and second pulleys are of about equal diameter.

3. The transmission of claim 1, wherein the first and second pulleys are of different diameters.

4. The transmission of claim 1, wherein the transmission link between the pulley housings is a pair of agonist and antagonist tendons wrapped in opposite directions about and between the pulley housings.

5. The transmission of claim 1, wherein the transmission link between the pulley housings is a pair of gears that each define teeth, wherein the teeth of each gear are engaged with the teeth of the other gear, thereby causing rotation of one of the pulley housings in one direction to rotate the other pulley housing in the opposite direction.

6. The transmission of claim 5, further including a cable housing coupled to each pulley housing and extending from each respective pulley.

7. The transmission of claim 6, further including the cable extending within the pulley housings, and across and between the pulleys, the cable further extending through the cable housings.

8. The transmission of claim 7, wherein each cable housing is rotatable about an axis coaxial to a major longitudinal axis of the cable extending within each respective cable housing.

9. The transmission of claim 1, further including a suspension handle at the ground link.

10. A tethered actuation system comprising:
a) an input mechanism;
b) an output mechanism;
c) a cable linking the input mechanism and the output mechanism;
d) at least one mechanical transmission, including
i) a ground link having first and second pivots that define parallel axes of rotation,
ii) a first pulley rotatable about the first pivot,
iii) a second pulley rotatable about the second pivot,
iv) a first pulley housing that rotates about the first pivot in response to a change in transmission angle of linear force of the cable at the first pulley, wherein the cable is threaded across and between the pulleys,
v) a second pulley housing that rotates about the second pivot in response to a change in transmission angle of linear force of the cable at the second pulley, and
vi) a transmission link between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of linear force across the mechanical transmission,
e) a first cable housing extending between the input mechanism and the at least one mechanical transmission; and
f) a second cable housing extending between the at least one mechanical transmission and the output mechanism.

11. The tethered actuation system of claim 10, wherein the input mechanism, the cable and the output mechanism constitute a Bowden cable system.

12. The tethered actuation system of claim 11, including two mechanical transmissions connected by the cable between the input mechanism and the output mechanism in parallel.

13. The tethered actuation system of claim 12, wherein each of the cable housings is rotatable about a major longitudinal axis of the cable extending within each of the cable housings.

14. The tethered actuation system of claim 10, wherein the first and second pulleys of the at least one mechanical transmission are of about equal diameter.

15. The tethered actuation system of claim 10, wherein the first and second pulleys of the at least one mechanical transmission are of different diameter.

16. The tethered actuation system of claim 10, wherein the transmission link between the pulley housings includes a pair of agonist and antagonist tendons wrapped in opposite directions about and between the pulley housings.

17. The tethered actuation system of claim 10, wherein the transmission link between the pulley housings includes a pair of gears that each define teeth, wherein the teeth of each gear are engaged with the teeth of the other gear, thereby causing rotation of one of the pulley housings in one direction to rotate the other pulley housing in the opposite direction.

18. The tethered actuation system of claim 17, wherein the cable housings are each attached to the pulley housings.

19. The tethered actuation system of claim 10, further including a control system in communication with the input mechanism and the output mechanism, the control system including:
a) a host computer that includes a user interface;
b) a master controller in communication with the host computer, the master controller providing real-time control and sensor fusion;
c) a local servo controller in communication with the master controller and the input mechanism, the local servo controller controlling the input mechanism;
d) sensors transmitting measurements of output states from the output mechanism; and
e) one or more input/output modules converting signals from the sensors and transmitting the converted signals to the master controller, whereby a torque command is produced and communicated to the input mechanism using measured feedback states from the sensors.

20. The tethered actuation system of claim 19, wherein the input mechanism transmits at least one of current and input angle feedback and emergency signals to the local servo controller.

21. The tethered actuation system of claim 20, wherein the input/output modules receive emergency signals from the output mechanism.

22. The tethered actuation system of claim 21, wherein the measured feedback states include at least one member of the group consisting of torque, angle, velocity and acceleration.

23. A method of actuating an end-effector, comprising the step of actuating an input mechanism, whereby force is transmitted from the input mechanism to an output mechanism through a cable that extends across at least one mechanical transmission, the at least one mechanical transmission including:
a) a ground link having first and second pivots that define parallel axes of rotation;
b) a first pulley rotatable about the first pivot;
c) a second pulley rotatable about the second pivot;
d) a first pulley housing that rotates about the first pivot in response to a change in transmission angle of linear force of a cable at the first pulley, the cable being threaded across and between the pulleys;
e) a second pulley housing that rotates about the second pivot in response to a change in transmission angle of linear force of the cable at the second pulley; and
f) a transmission link between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of linear force across the mechanical transmission.

24. An ankle exoskeleton system design, comprising:
a) an electric motor;
b) an input mechanism;
c) an output mechanism;
d) a cable linking the input mechanism and the output mechanism;
e) at least one mechanical transmission, including
i) a ground link having first and second pivots that define parallel axes of rotation,
ii) a first pulley rotatable about the first pivot,
iii) a second pulley rotatable about the second pivot,
iv) a first pulley housing that rotates about the first pivot in response to a change in transmission angle of linear force of a cable at the first pulley, the cable threaded across and between the pulleys,
v) a second pulley housing that rotates about the second pivot in response to a change in transmission angle of linear force of the cable at the second pulley, and
vi) a transmission link between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of linear force across the mechanical transmission;
f) a first cable housing extending between the input mechanism and the at least one mechanical transmission; and
g) a second cable housing extending between the at least one mechanical transmission and the output mechanism.

25. The ankle exoskeleton system design of claim 24, including a first mechanical transmission and a second mechanical transmission connected by the cable in series.

26. The ankle exoskeleton system design of claim 25, further including a harness to which the first and second mechanical transmissions are connected, wherein the first mechanical transmission is fixed proximate to a human hip joint, and the second mechanical transmission is fixed proximate to a knee joint of a human subject.

27. A wearable device, comprising:
a) a distal member wearable by an individual distal to a skeletal joint of the individual;
b) a proximal member including a tube, an actuator and a harness, wearable by the individual proximal to the joint, wherein one or the other of the distal member and the proximal member includes an elastic crossing member;
c) a link between the distal member and the proximal member, wherein the elastic crossing member and the link span an axis about which the distal member rotates, from one to the other of the distal member or the proximal member, and whereby actuation of the link is translated to a force at the distal or proximal member that is normal to a major longitudinal axis extending through the distal and proximal members;
d) a cable connected to the crossing member and extending from the crossing member to the actuator; and
e) at least one mechanical transmission between at least one of: the distal member and the proximal member; and the actuator and the tube, the mechanical transmission including
i) a ground link having first and second pivots that define parallel axes of rotation,
ii) a first pulley rotatable about the first pivot,
iii) a second pulley rotatable about the second pivot,
iv) a first pulley housing that rotates about the first pivot in response to a change in transmission angle of linear force at the first pulley of a cable threaded across and between the pulleys,
v) a second pulley housing that rotates about the second pivot in response to a change in transmission angle of linear force at the second pulley of the cable, and
vi) a transmission link between the pulley housings, whereby rotation of one of the pulley housings in one direction causes rotation of the other pulley housing in the opposite direction, thereby causing the first and second pulley housings to rotate about the first and second pivots, respectively, of the ground link, in response to a change in transmission angle of linear force across the mechanical transmission.

28. The device of claim 27, wherein the link includes a strut, the strut extending from the proximal member to the distal member.

29. The device of claim 28, wherein the strut is constrained at the proximal member normally and laterally to a major longitudinal axis of the crossing member extending from the proximal member to the distal member, wherein the strut is not restricted along the major longitudinal axis of the crossing member.

30. The device of claim 29, wherein the link further includes at least one roller at the proximal member that constrains the strut normally and laterally.

31. The device of claim 30, wherein the link includes at least one pair of rollers in opposition to each other, wherein the strut is normally constrained between the pair of rollers.

32. The device of claim 31, wherein the strut is curved at the pair of rollers, whereby shear force between the strut and the pair of rollers during rotation of the distal member about the axis spanned by the crossing member and the strut is less than it would be if the strut were straight at the pair of rollers.

33. The device of claim 32, wherein the strut includes a guide tube at the pair of rollers, wherein the crossing member extends through the guide tube.

34. The device of claim 33, including a pair of crossing members and a pair of struts.

35. The device of claim 34, wherein the struts are essentially straight between the rollers and the distal member.

36. The device of claim 35, wherein at least one of the struts deflects during eversion and inversion of a human foot secured to the distal member and a human calf secured to the proximal member.

37. The device of claim 36, wherein the struts are rigid.

38. The device of claim 34, wherein the struts are curved, whereby the struts operate as series springs during a normal walking cycle of a human foot secured to the distal member and a human calf secured to the proximal member.

39. The device of claim 34, wherein the link further includes a motor actuator assembly attached to a proximal end of the pair of crossing members, whereby actuation of the link will cause retraction of the crossing members, which causes rotation of the distal member and plantar flexion of a human foot secured to the distal member about a human ankle joint.

40. The device of claim 34, wherein the pair of crossing members is fixed to a proximal end of the distal member.

41. The mechanical transmission of claim 1, wherein the length of cable is a first length of cable, and further including a third pulley rotatable about the first pivot and a fourth pulley rotatable about the second pivot, whereby a second length of cable can extend across and between the third and fourth pulleys.

42. The mechanical transmission of claim 41, wherein the first length of cable and the second length of cable extend across and between the first and second pulleys, and the third and fourth pulleys, respectively, in opposite directions, whereby the first and second lengths of cable cross each other at a centerline between the axes of rotation of the first and second pulleys, and the third and fourth pulleys, respectively.

43. The mechanical transmission of claim 41, wherein the first length of cable and the second length of cable extend across and between the first and second pulleys, and the third and fourth pulleys, respectively, in the same direction, whereby the first and second lengths of cable are essentially parallel to each other at a centerline between the axes of rotation of the first and second pulleys, and the third and fourth pulleys, respectively.

44. The mechanical transmission of claim 41, further including an adapter fixed to the second pulley housing, wherein the adapter defines a first axis that is parallel to the axis of rotation of the first pivot, and a second axis is transverse to the axis of rotation of the first pivot.

45. The mechanical transmission of claim 44, wherein the second axis is normal to the first axis in a plan view of the first and second axes.

46. The mechanical transmission of claim 45, further including
a) a second ground link defining a third pivot and fourth pivot defining distinct axes of rotation parallel to the second axis;
b) a fifth pulley rotatable about the third pivot;
c) a sixth pulley rotatable about the fourth pivot;
d) a seventh pulley rotatable about the third pivot;
e) an eighth pulley rotatable about the fourth pivot;
f) a third pulley housing that rotates about the third pivot in response to a change in transmission angle of linear force at the fifth and seventh pulleys of either or both of the first and second lengths of cable threaded across and between the fifth and seventh pulleys, wherein the third pulley housing is fixed to the adapter;
g) a fourth pulley housing that rotates about the fourth pivot in response to a change in transmission angle of linear force at the sixth and eighth pulleys of either or both of the first and second lengths of cable threaded across and between the sixth and eighth pulleys; and
h) a transmission link between the third and fourth pulley housings, whereby rotation of one of the third and fourth pulley housings in one direction causes rotation of the other of the third and fourth pulley housings in the opposite direction, thereby causing the third and fourth pulley housings to rotate about the third and fourth pivots, respectively, of the second ground link, in response to a change in transmission angle of linear force of the cable across the third and fourth pivots.

47. The mechanical transmission of claim 1, further includes:
a) an adapter fixed to the second pulley housing, wherein the adapter defines a first axis that is parallel to the axis of rotation of the first pivot, and a second axis is transverse to the axis of the rotation of the first pivot;
b) a second ground link defining a third pivot and fourth pivot, the third and fourth pivots, defining distinct axes of rotation parallel to the second axis;
c) a third pulley rotatable about the third pivot;
d) a fourth pulley rotatable about the fourth pivot;
e) a third pulley housing that rotates about the third pivot in response to a change in transmission angle of linear force of the cable threaded across and between the third and fourth pulleys, the third pulley housing being attached to the adapter;
f) a fourth pulley housing that rotates about the fourth pivot in response to a change in transmission angle of linear force of the cable at the fourth pulley; and
g) a transmission link between the third and fourth pulley housings, whereby rotation of one of the third and fourth pulley housings in one direction causes rotation of the other of the third and fourth pulley housing in the opposite direction, thereby causing the third and fourth pulley housings to rotate about the third and fourth pivots, respectively, of the second ground link, in response to a change in transmission angle of linear force across the third and fourth pivots.
